(19) United States
(12) Patent Application Publication
(10) Pub. No.: US 2026/0294878 A1
(43) Pub. Date: Oct. 1, 2026

(54) INJECTABLE, SUSTAINED-RELEASE MICROSPHERE FORMULATIONS CONTAINING RAPAMYCIN AND/OR RAPAMYCIN ANALOGS AND METHODS OF MAKING AND USING THE FORMULATIONS

(71) Applicant: Texas Animal Health Innovations LLC, Angleton, TX (US)

(72) Inventors: Dana M. Vaughn, Seguin, TX (US); Rachel Minrovic, Willoughby, OH (US); Stephen Ashbaugh, Cleveland, OH (US); Tracy Richey, Stow, OH (US)

(21) Appl. No.: 19/701,597

(22) Filed: Jun. 8, 2026

Related U.S. Application Data
(63) Continuation of application No. 19/578,295, filed on Mar. 25, 2026.
(60) Provisional application No. 63/778,175, filed on Mar. 26, 2025.

(51) Int. Cl. A61K 31/436; A61K 9/00; A61K 9/16; A61P 37/02

(52) U.S. Cl. CPC A61K 31/436; A61K 9/0019; A61K 9/1647; A61P 37/02

(57) ABSTRACT

A microsphere formulation is provided, as well as a pharmaceutical composition including the microsphere formulation, a kit for forming the pharmaceutical composition and a therapeutic regimen including administering the pharmaceutical composition to a subject to treat an immune-mediated disease. The microsphere formulation includes polymer microspheres, wherein each polymer microsphere includes: an active pharmaceutical ingredient consisting of rapamycin, a rapamycin analog, a pharmaceutically acceptable salt of rapamycin, and/or a pharmaceutically acceptable salt of the rapamycin analog; and a biodegradable polymer, wherein each polymer microsphere comprises a drug load of the active pharmaceutical ingredient that is between 20 and 40 percent by weight of the polymer microsphere, and wherein the polymer microspheres have a median particle size of about 15 micrometers to about 30 micrometers.

### BACKGROUND

The present disclosure relates to formulations containing rapamycin, methods of making formulations containing rapamycin, and methods of treating subjects using formulations containing rapamycin.

### BACKGROUND OF THE RELATED ART

Rapamycin, also known as sirolimus, is a macrolide compound originally discovered in the soil of Easter Island (Rapa Nui) and developed as an immunosuppressant drug. It primarily works by inhibiting the mechanistic target of rapamycin (mTOR), a key regulatory protein involved in cell growth, proliferation, and metabolism. Rapamycin is a macro cyclic lactone antibiotic, meaning its primary structure consists of a large, ring-shaped molecule with a lactone group (a cyclic ester) within the ring. Rapamycin is characterized by a 29-membered ring containing multiple trans double bonds, its chemical formula is C₅₁H₇₉NO₁₃, and it has a molecular weight of 914.2 g/mol. Rapamycin may be identified by a CAS (Chemical Abstract Service) Number 53123-88-9, a CID (PUBMED compound identifier) 5284616 and 5497196, and/or the IUPAC (International Union of Pure and Applied Chemistry) Name: (1R,9S,12S,15R,16E,18R,19R,21R,23S,24E,26E,28E,30S,35R)-1,18-dihydroxy-12-[(2R)-1-[(1S,3R,4R)-4-hydroxy-3-methoxycyclohexyl]propan-2-yl]-19,30-dimethoxy-15,17,21,23,29,35-hexamethyl-11,36-dioxa-4-azatricyclo[30.3.1.04,9]hexatriaconta-16,24,26,28-tetraene-2,3,10,14,20-pentone. Rapamycin is characterized by the general structure:

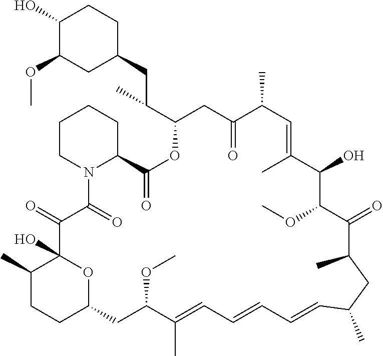 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Rapamycin has been used to treat a variety of conditions, including immune-mediated and autoimmune diseases. The commonality between autoimmune diseases and immune-mediated diseases is that they both involve a malfunctioning immune system, where the body's defense systems either attack healthy tissues as in autoimmune diseases, or fails to adequately protect against infections as in immunity diseases leading to abnormal immune responses and potential chronic health complications.

A cellular commonality between immune-mediated and autoimmune diseases is the dysregulation or overactivation of plasmocytic and lymphocytic cells. The cell types primarily include plasmocytic dendritic cells, and T lymphocytes, B lymphocytes and Natural Killer cells. All these cell types can have overactive mTOR complexes that can activate kinases which phosphorylate activating metabolic processes that can result in a variety of disease conditions. Both types of diseases can cause inflammation, are generally refractory to antibiotic treatments and can have symptoms that come and go.

Different autoimmune and immune-mediated diseases affect various parts of the body. However, systemic involvement is commonly seen in all these conditions, causing significant mortality and morbidity. It is well-known that both environmental factors including diet, drugs, geographical, social status stress, infectious agents and genetics can play a role in the pathogenesis of these conditions. These diseases pose significant systemic medical challenges and are often accompanied by various co-morbidities. Despite existing treatments, the conditions can be challenging to manage, and many require lifelong care to improve quality of life. These types of diseases may have acute flareups but are generally characterized by having a chronic underlying disease.

Rapamycin may also provide therapeutic benefits by inducing the cleaning out of accumulated cellular debris by activating the mechanisms of autophagy. Rapamycin induces autophagy by inhibiting a central cellular growth regulator called mTORC1 (mechanistic target of rapamycin complex 1). Under normal conditions, mTORC1 suppresses autophagy by blocking the activation of proteins required to initiate autophagosome formation. When rapamycin binds to the protein FKBP12, the resulting complex directly inhibits mTORC1, lifting this suppression. As mTORC1 activity falls, upstream autophagy-initiating kinases such as ULK1 become activated, allowing the cell to begin assembling the autophagy machinery. This shift promotes the formation of autophagosomes, enhances lysosomal biogenesis, and ultimately increases the degradation and recycling of cellular components. In essence, rapamycin pushes the cell into a resource-conserving, self-cleaning mode by turning down growth signals and turning up catabolic pathways.

Rapamycin has been shown to improve skin barrier function by inducing autophagy, which is crucial for maintaining cellular health and preventing the accumulation of damaged proteins and other molecules associated with age-related diseases and tissue dysfunction. By promoting autophagy, rapamycin treatment enhances the health and function of the skin layer by increasing the clearance of damaged cellular components and byproducts. This has been demonstrated in studies where rapamycin treatment led to a significant reduction in P16 expression, indicating a diminished number of senescent cells in the epidermis. Additionally, rapamycin treatment has been associated with improved distribution of cytokeratin 5/6 in the basal layer, which is vital for the development and maintenance of healthy skin. These findings suggest that rapamycin may have a broader impact on the maintenance and repair of the skin, providing additional benefits beyond reducing markers of aging and preventing sun damage.

Rapamycin has been shown to ameliorate kidney fibrosis by inhibiting the activation of mTOR signaling in interstitial macrophages and myofibroblasts. This action reduces the interstitial infiltrates and accumulation of extra cellular matrix (ECM), which are key factors in the development of kidney fibrosis. Rapamycin may be effective in preventing kidney fibrosis by blocking the mTOR signaling pathway in these cells.

### BRIEF SUMMARY

Some embodiments provide a microsphere formulation comprising polymer microspheres, wherein each polymer microsphere comprises: an active pharmaceutical ingredient consisting of rapamycin, a rapamycin analog, a pharmaceutically acceptable salt of rapamycin, and/or a pharmaceutically acceptable salt of the rapamycin analog; and a biodegradable polymer, wherein each polymer microsphere comprises a drug load of the active pharmaceutical ingredient that is between 20 and 40 percent by weight of the polymer microsphere, and wherein the polymer microspheres have a median particle size of less than or equal to 100 micrometers.

Some embodiments provide a pharmaceutical composition comprising a microsphere formulation comprising polymer microspheres, wherein each polymer microsphere comprises: an active pharmaceutical ingredient consisting of rapamycin, a rapamycin analog, a pharmaceutically acceptable salt of rapamycin, and/or a pharmaceutically acceptable salt of the rapamycin analog; and a biodegradable polymer comprising an acid-terminated poly(lactide-co-glycolide) polymer having a lactide:glycolide ratio of about 50:50, wherein the biodegradable polymer has an inherent viscosity between about 0.1 and about 0.3 deciliters per gram, wherein each polymer microsphere comprises a drug load of the active pharmaceutical ingredient that is between 25 and 40 percent by weight of the polymer microsphere, and wherein the polymer microspheres have a median particle size of less than or equal to 30 micrometers. The pharmaceutical composition may further comprise a diluent, such as a diluent including mannitol, sodium carboxymethylcellulose, and polysorbate 80.

Some embodiments provide a therapeutic regimen for treating an immune-mediated disease in a subject using a microsphere formulation. The therapeutic regimen comprises administering an injection of a pharmaceutical composition comprising the microsphere formulation and a diluent, wherein the pharmaceutical composition provides a therapeutically effective blood concentration of the active pharmaceutical ingredient greater than 5 nanograms per milliliter of blood of the subject over a period of 14 days. The microsphere formulation comprises polymer microspheres, wherein each polymer microsphere comprises: an active pharmaceutical ingredient consisting of rapamycin, a rapamycin analog, a pharmaceutically acceptable salt of rapamycin, and/or a pharmaceutically acceptable salt of the rapamycin analog; and a biodegradable polymer comprising an acid-terminated poly(lactide-co-glycolide) polymer having a lactide:glycolide ratio of about 50:50, wherein the biodegradable polymer has an inherent viscosity between about 0.1 and about 0.3 deciliters per gram, wherein each polymer microsphere comprises a drug load of the active pharmaceutical ingredient that is between 25 and 40 percent by weight of the polymer microsphere, and wherein the polymer microspheres have a median particle size of less than or equal to 30 micrometers.

Some embodiments provide a kit comprising a microsphere formulation comprising polymer microspheres, wherein each polymer microsphere comprises: an active pharmaceutical ingredient consisting of rapamycin, a rapamycin analog, a pharmaceutically acceptable salt of rapamycin, and/or a pharmaceutically acceptable salt of the rapamycin analog; and a biodegradable polymer comprising an acid-terminated poly(lactide-co-glycolide) polymer having a lactide:glycolide ratio of about 50:50, wherein the biodegradable polymer has an inherent viscosity between about 0.1 and about 0.3 deciliters per gram, wherein each polymer microsphere comprises a drug load of the active pharmaceutical ingredient that is between 25 and 40 percent by weight of the polymer microsphere, and wherein the polymer microspheres have a median particle size of less than or equal to 30 micrometers. The kit may further comprise a separate amount of a diluent to be added to the microsphere formulation to form a pharmaceutical composition prior to administration to a subject via injection.

### BRIEF DESCRIPTION OF THE SEVERAL VIEWS OF THE DRAWINGS

FIG. 1 is a High-Performance Liquid Chromatography (HPLC) chromatogram after mixing in a levitating magnetic impeller pump or mixer illustrating the preservation of rapamycin (sirolimus) and avoidance of generating seco-rapamycin.

FIG. 2 is a High-Performance Liquid Chromatography (HPLC) chromatogram of rapamycin (sirolimus) after exposure to heat illustrating a significant degradation of rapamycin to seco-rapamycin.

FIG. 3 is graph of the in vivo rapamycin blood level (ng/ml) over a period of 21 days after administration of three different extended-release rapamycin formulations.

### DETAILED DESCRIPTION

Some embodiments provide a microsphere formulation comprising polymer microspheres, wherein each polymer microsphere comprises: an active pharmaceutical ingredient consisting of rapamycin, a rapamycin analog, a pharmaceutically acceptable salt of rapamycin, and/or a pharmaceutically acceptable salt of the rapamycin analog; and a biodegradable polymer comprising an acid-terminated poly(lactide-co-glycolide) polymer having a lactide:glycolide ratio of about 50:50, wherein the biodegradable polymer has an inherent viscosity of about 0.1 to about 0.3 deciliters per gram, wherein each polymer microsphere comprises a drug load of the active pharmaceutical ingredient that is between 20 and 40 percent by weight of the polymer microsphere, and wherein the polymer microspheres have a median particle size of less than or equal to 100 micrometers.

Some embodiments provide an injectable, sustained-release (SR) formulation containing rapamycin and/or a rapamycin analog that is beneficial for administration to an animal, such as a veterinary species or a human, to treat one or more conditions being experienced by the animal. After injection into the animal (also referred to as the “subject”), the formulation provides a sustained release of the rapamycin and/or rapamycin analog. Both rapamycin and rapamycin analogs are considered to be mTOR inhibitors (“mammalian target of rapamycin” or “mechanistic target of rapamycin”). Without limitation, the animal may be a cat, dog, horse, livestock, zoological specimen, wildlife, other veterinary animals, or humans.

The term “sustained release” refers to a drug delivery formulation having one or more components designed to release an active ingredient (the “drug”) gradually over an extended period, preferably ensuring a consistent therapeutic drug level and effect while minimizing the frequency of dosing. Unlike immediate-release formulations, which release the active ingredient quickly, sustained-release drugs may be engineered to maintain a steady level of the drug in the bloodstream, reducing peaks (high concentrations or levels) and troughs (low concentrations or levels) that can lead to side effects or suboptimal efficacy. This controlled release is achieved through specialized formulation techniques, such as embedding the drug in a matrix or coating the drug with a substance that dissolves slowly. Sustained-release systems enhance compliance with administration schedules by reducing the frequency of dosing and can improve the overall effectiveness of treatment by maintaining stable drug concentrations over time.

Rapamycin and its derivatives or analogs are inhibitors of mTOR. These drugs are approved for use in anticancer therapies, rejection prophylaxis after organ transplant, drug-eluting coronary stents, and the treatment of Lymphangiolemyomatosis and tuberous sclerosis. Rapamycin is currently orally administered as an ingestible tablet, ingestible liquid or surgically inserted as a stent. Rapamycin is commercially available as a generic, and under the trade name Sirolimus and is sold under the brand name of RAPAMUNE® (a registered trademark of Pfizer) Sirolimus (RAPAMUNE®) is a once daily ingestible tablet or liquid having a dose of 2 and 5 mg. However, long term maintenance treatment through this route is problematic as some animals, such as cats and dogs, experience problems with daily oral dosing for the treatment of chronic immune-mediated diseases that require daily administration. Furthermore, some rapamycin inhibitors may have low and variable oral bioavailability. For example, rapamycin may have an oral bioavailability of only 15% in the fasted state, but this can vary from one subject or patient to another.

Some embodiments provide a highly bioavailable, sustained-release formulation comprising rapamycin and/or rapamycin analogs that may be administered by an injection, without the need for administration of closely timed, successive dosages under supervision from a medical professional. For example, the injectable formulation may include sustained-release rapamycin-encapsulating microspheres. In one option, the microspheres provide a high drug load (>20% rapamycin/analogs by total weight of the microspheres), a small particle size (a median (D₅₀) of about 15-60 micrometers (μm), a relatively low molecular weight polymer(s), a low initial burst release and long release duration (≥^{~}7, 15, 30, 60, 90, 120, or even 360 days).

In addition to rapamycin itself, inhibitors of mTOR also include rapamycin analogs. Non-limiting examples of rapamycin analogs include, but are not limited to, everolimus, tacrolimus, CC1-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-emethoxy-rapamycin, 2-desmethyl-rapamycin, and 42-O-(2-hydroxy)ethyl rapamycin. Other rapamycin analogs include: rapamycin oximes; rapamycin aminoesters; rapamycin dialdehydes; rapamycin 29-enols, O-alkylated rapamycin derivatives, water soluble rapamycin esters, alkylated rapamycin derivatives, rapamycin amidino carbamates, biotin esters of rapamycin, carbamates of rapamycin, rapamycin hydroxyesters, rapamycin 42-sulfonates and 42-(N-carboalkoxy) sulfamates, rapamycin oxepane isomers, imidazolidyl rapamycin derivatives, rapamycin alkoxyesters, rapamycin pyrazoles, acyl derivatives of rapamycin, reduction products of rapamycin, rapamycin amide esters, rapamycin fluorinated esters, rapamycin acetals, oxorapamycins, and rapamycin silyl ethers. Rapamycin and/or the rapamycin analogs used in the formulations may be obtained from any known source including artificial and/or natural sources. Rapamycin and rapamycin analogs may be chemically synthesized using any known technique. Furthermore, it should be understood that references to rapamycin herein are also directed to rapamycin analogs and/or other mTOR inhibitors unless the context of the reference is clearly limited to rapamycin.

### Formulation Processes

Some embodiments provide a method for treating an immune-mediated disease in an animal. The method may comprise administering to an animal (patient or subject) in need thereof a microsphere formulation, the microsphere formulation comprising: polymer microspheres, each polymer microsphere comprising: (i) rapamycin; and (ii) a PLGA polymer, wherein each polymer microsphere comprises a drug load of between about 20 wt/wt % to about 40 wt/wt %, and wherein the polymer microspheres have a median particle size (D₅₀) from about 15 μm to about 60 μm (D₅₀). In one option, the microsphere formulation may be administered to the animal by intramuscular or subcutaneous injection. In another option, a dosing schedule of about every seven to fifteen days may be implemented.

Some embodiments provide a microsphere formulation comprising rapamycin and/or rapamycin analog. The microsphere formulation comprises polymer microspheres, each polymer microsphere comprising: (i) rapamycin; and (ii) a biodegradable polymer, wherein each polymer microsphere comprises a drug load of the rapamycin greater than 20% by weight of the polymer microsphere, and wherein the polymer microspheres have a median particle size (D₅₀) of less than 100 μm. Optionally, the median particle size (D₅₀) of the polymer microspheres may be less than 60 μm or less than 30 μm.

Some embodiments provide a method for treating immune mediated diseases in an animal, which may include a veterinary animal and/or a human. The method may comprise administering a microsphere formulation by subcutaneous, intramuscular or intra-articular injection to an animal in need thereof, wherein the microsphere formulation is made according to the methods described herein and/or has a composition as described herein, and wherein the formulation is administered to the animal with a dosing schedule ranging between weekly and annually, such as a dosing schedule of about every 7, 15, 30, 60, 90, 120 or 365 days.

In some embodiments, the microsphere formulation may be characterized in that the rapamycin is released over a period ranging from about one week to about one year. In one option, microsphere formulation may be characterized in that the rapamycin is released over a period ranging from about 7 to about 30 days, from about 7 to about 15 days, or from about 15 to about 30 days. In another option, the microsphere formulation may be characterized in that the rapamycin is released over a period ranging from about 60 to about 90 days. In yet another option, the microsphere formulation may be characterized in that the rapamycin is released over a period ranging from about 120 to about 365 days.

In some embodiments, the microsphere formulation may be characterized in that the microspheres have a low initial burst release. In one example, the initial burst release may release no more than 20% of the rapamycin within about 24 hours following injection into an animal.

In some embodiment, any microsphere formulation disclosed herein may be sterilized by irradiation prior to administration.

Some embodiments provide a kit, the kit comprising polymer microspheres, each polymer microsphere comprising: (i) an active pharmaceutical ingredient consisting of rapamycin, a rapamycin analog, a pharmaceutically acceptable salt of rapamycin, and/or a pharmaceutically acceptable salt of the rapamycin analog; and (ii) a biodegradable polymer, wherein each polymer microsphere comprises a drug load of the active pharmaceutical ingredient that is between 20 and 40 percent by weight of the polymer microsphere, and wherein the polymer microspheres have a median particle size of less than or equal to 100 micrometers.

Some embodiments provide a microsphere formulation comprising rapamycin and/or rapamycin analogs. The microsphere formulation comprises polymer microspheres, each polymer microsphere comprising: (i) rapamycin; and (ii) a biodegradable polymer comprising either a poly(lactide-co-glycolide) polymer (a “PLGA”) or a poly(D,L-lactide) polymer (a “PLA”), wherein each polymer microsphere comprises a drug load of rapamycin of greater than 20% by weight of the polymer microsphere, and wherein the polymer microspheres have a median particle size (D₅₀) from about 15 μm to about 60 μm.

TABLE 1
General Parameters for Making the Microspheres

| Formulation Parameter | Current or Intended Range |
|---|---|
| Polymer type | PLGA or PLA (preferentially PLGA - |
| | 50:50 - 75:25 L:G) |
| Polymer end group | Ester or acid |
| Polymer Inherent | <0.5 dL/g (preferentially <0.3 dL/g; |
| Viscosity (IV) | such as 0.12 DL/g) |
| Solvent System | Dichloromethane |
| Particle Size | <100 μm (preferentially <50 μm) |
| Drug Loading | 20-40 wt % (may go up to 50 wt %) |

### Preparation of the Sustained Release Formulation

Some embodiments provide a method for producing microspheres or microparticles using a homogenizer system or apparatus used for contacting the dispersed phase and the continuous phase. As one example, the homogenizer may be a Levitronix® BPS-i100 integrated pump system used, e.g., as described in U.S. Pat. No. 11,167,256, which is incorporated by reference herein in its entirety. The Levitronix® BPS-i100 is a levitating magnetic impeller pump containing a levitating impeller within the chamber of the pump that is not in contact with any surface. Rather, the levitating impeller is rotated using magnetism. More specifically, a rotating magnetic field is created outside of the sealed chamber and is directed to the inside of the chamber, causing levitation and rotation of the impeller. The use of magnetic forces enables the formation of microspheres in the sealed pump chamber that is free of contacting parts present in rotor stator homogenizers, because no such rotors or other such parts are present in the chamber, but rather only the levitating impeller driven by the magnetic forces is contained within the chamber. As such, the microspheres are formed while foreign particulate matter is eliminated, or at least greatly reduced as compared to manufacturing of microspheres with a rotor stator homogenizer. A levitating magnetic impeller pump that is designed to create a low shear environment within its pumping chamber may be made to act as a homogenizer producing a high shear environment within its chamber by operating the pump in a manner that is opposite its natural direction of flow. This opposite operation may be achieved by pumping a continuous phase and dispersed phase through the levitating magnetic impeller pump in the opposite direction as intended by the manufacturer. As another example, the homogenizer may be a Silverson in-line homogenizer.

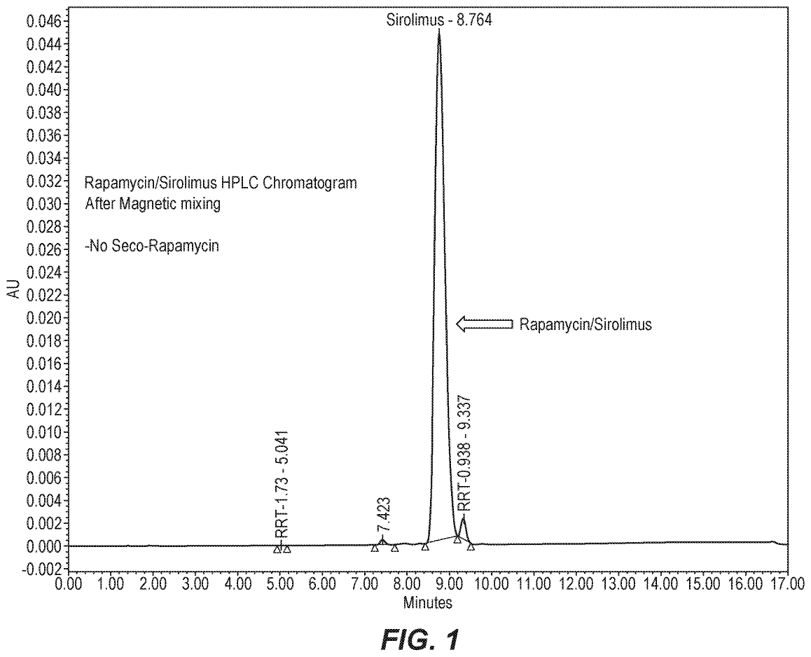

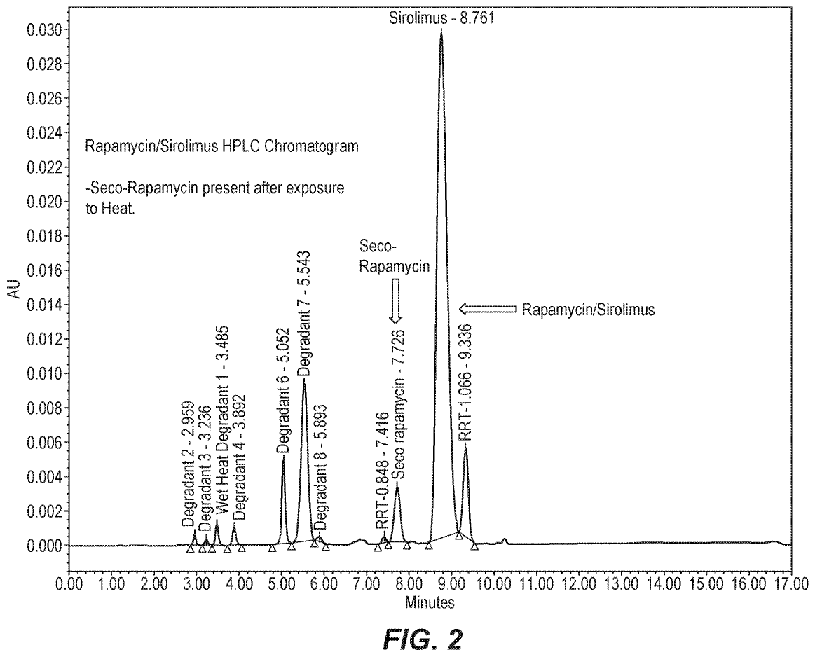

A novel finding of the overall manufacturing process is that the rapamycin maintains its integrity in a way that rapamycin does not degrade into secondary undesirable metabolites, such as seco-rapamycin (FIG. 1) during manufacturing. Exposure to heat induces the rapamycin structure to degrade and form undesirable degradants, such as seco-rapamycin (FIG. 2). The encapsulation of rapamycin into the biodegradable polymers appears to protect the rapamycin molecule from degradation into seco-rapamycin under conditions that would normally be expected to produce seco-rapamycin. For example, the microspheres may be washed with water at about 40° C. without degradation of the microsphere-encapsulated rapamycin, whereas rapamycin that is not encapsulated will begin to degrade at 40° C.

Some embodiments provide a method for producing microspheres or microparticles using a homogenizer; wherein a dispersed phase liquid or dispersed phase suspension (organic phase) is pumped into homogenizer; wherein a continuous phase (aqueous phase) liquid is pumped into the homogenizer; wherein the ratio of aqueous phase to the dispersed phase is between 1:1 and 1:80, and wherein the dispersed phase liquid and the continuous phase liquid are homogenized in a shear environment created by the homogenizer to form microspheres.

In some embodiments, the dispersed phase may comprise a variety of solutions comprised of polymers, active pharmaceutical ingredients, and organic solvents. Exemplary pharmaceutical ingredients that may be used include sirolimus (rapamycin) and the rapalog derivatives, analogs, or isomers related to rapamycin. Exemplary polymeric compounds that may be used include poly(lactic-co-glycolic acid) (PLGA), PLGA-PEG (PLGA and polyethylene glycol co-polymer), PEG (polyethylene glycol), cellulosic polymers, polycaprolactone, polyglycolide, polylactic acid (PLA), poly-3-hydroxybutyrate, polyhydroxyalkanoates, polyesteramide (PEA), polyanhydrides, polyacetals, poly(ortho esters), polyphosphoesters, polyureas, polycarbonates, thermoplastic polyolefins (TPO) and polyolefin elastomers (POE). The continuous phase may comprise at least water. Optionally, a surfactant may be included. Examples of such optional surfactants may include polyvinyl alcohol (PVA), polyvinylpyrrolidone (PVP), calcium stearate (CSt), and methyl cellulose. The materials used for the impeller and the housing of the pump chamber are, in one aspect, preferably biocompatible (i.e. compliant with FDA and USP-VI, ABSE/TSE and animal free) resins. In such an arrangement, it can be ensured that no such non-biocompatible material is introduced to the microsphere product.

### EXAMPLES

Example 1: Three batches of microspheres were prepared according to the parameters set out in the Table below. For each batch, the steps used to prepare the microspheres were the same. These steps included: (1) dissolve polymer and rapamycin in dichloromethane (DCM; also known as methylene chloride); (2) filter using a 0.2 μm membrane filter made with polytetrafluoroethylene (PTFE) or polyvinylidene fluoride (PVDF); (3) Pump the dispersed phase (DP) and the continuous phase (CP) (water and 0.35% PVA) into a homogenizer at a set flow rate with the homogenizer set to a target speed to manufacture microspheres at a specific size distribution; (4) microspheres flow from the homogenizer to a solvent removal vessel (SRV); (5) water may also be added to the SRV to minimize solvent levels; (5) solvent is removed and the microspheres are washed using a hollow fiber filter (HFF). A representative “downstream” microsphere processing phase is described in U.S. Pat. No. 6,270,802, which is incorporated by reference herein in its entirety; and (6) microspheres are collected and freeze dried using a lyophilizer to form a free-flowing bulk powder.

TABLE 2
Specific Parameters Used to Make Three Batches of
Microspheres Containing Rapamycin for Example 1

| Batch Number | 7 | 12 | 14 |
|---|---|---|---|
| Polymer Type | PLGA | | |
| Co-Monomer Ratio | 50:50 | | |
| Polymer IV (dL/g) | 0.13 | | |
| Polymer End Cap | Acid | | |
| Solvent System | DCM | | |
| Target Drug Load | 25 | 40 | 25 |
| Actual Drug Load | 24.6 | 40.5 | 25.3 |
| Homogenizer RPM | 3000 | | 5000 |
| Particle Size (D₁₀) | 15 | 17 | 7 |
| Particle Size (D₅₀) | 29 | 29 | 15 |
| Particle Size (D₉₀) | 49 | 50 | 33 |
| Residual Solvents (% wt.) | 0.1 | 0.1 | 0.01 |
| Polymer MW (KDa) | 8.6 | 7.8 | 7.8 |
| Microsphere MW (kDa) | 7.1 | 6.3 | 6.4 |

The three batches of microspheres described in Table 2 were used in a pharmacokinetic (PK) study to examine the in vivo release of rapamycin in rats. Each batch of microspheres was evaluated on the ability of the microspheres to provide a therapeutic level of greater than 5 nanograms rapamycin per milliliter of blood in rats over a period of 7, 14 and 21 days.

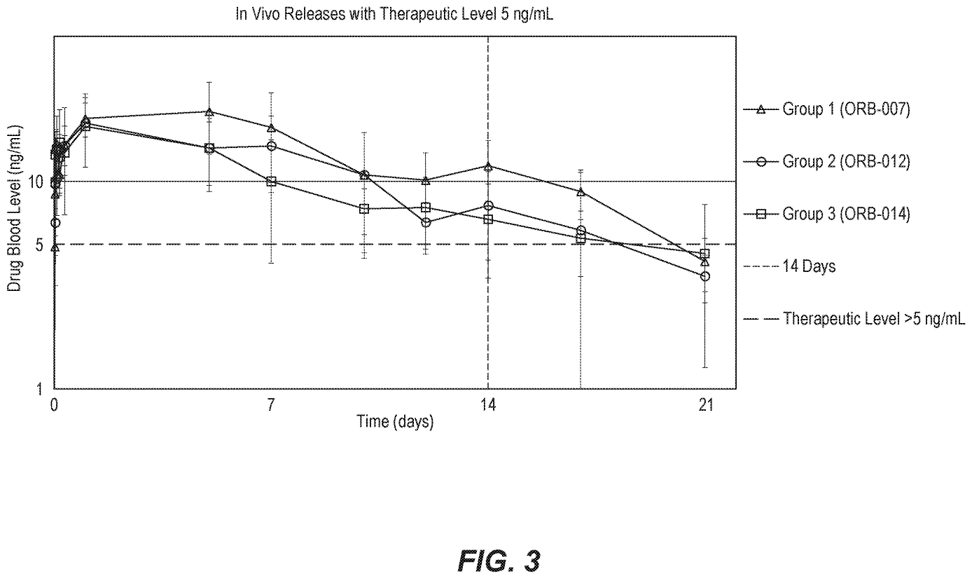

FIG. 3 is graph of the in vivo rapamycin blood level (ng/mL) in rats over a period of 21 days after subcutaneous administration of three different extended-release rapamycin formulations. Administration of all 3 formulations resulted in no initial large burst release of rapamycin. All 3 formulations continued to maintain over 5 ng/ml whole blood levels of rapamycin (a reported minimal targeted immuno-therapeutic blood level of rapamycin) for 17 days, followed by levels above 1 ng/ml throughout 21 days. The ORB-007 formulation provided consistently higher rapamycin blood levels than did ORB-012 or ORB-014 through 14 and 17 days.

Example 2: Following the general procedure described in Example 1, three additional microsphere batches were prepared using the parameters in Table 3, below.

TABLE 3
Specific Parameters Used to Make Three Batches of
Microspheres Containing Rapamycin for Example 2

| | | | |
|---|---|---|---|
| Batch Number | 8 | 9 | 11 |

| | | |
|---|---|---|
| Polymer Type | PLGA | PLA |

| | | | |
|---|---|---|---|
| Co-Monomer Ratio | 50:50 | 75:25 | 100:0 |
| Polymer IV (dL/g) | 0.13 | 0.12 | 0.13 |

| Polymer End Cap | Acid | |
|---|---|---|
| Solvent System | DCM | |
| Target Drug Load | 25 | 40 |

| | | | |
|---|---|---|---|
| Actual Drug Load | 25.0 | 39.8 | 39.4 |

| | | |
|---|---|---|
| Homogenizer RPM | 1500 | 3000 |

| | | | |
|---|---|---|---|
| Particle Size (D₁₀) | 39 | 18 | 101 |
| Particle Size (D₅₀) | 64 | 30 | 48 |
| Particle Size (D₉₀) | 101 | 30 | 49 |
| Residual Solvents (% wt.) | 0.93 | 0.29 | 0.27 |
| Polymer MW (KDa) | 7.1 | 8.0 | 9.1 |
| Microsphere MW (kDa) | 8.1 | 9.0 | 9.9 |

### Rapamycin Dosing Regimens and Duration of Dosing.

Indications and usage: Sustained-release Rapamycin (SR-Rapamycin) is an mTOR inhibitor immunosuppressant indicated for the treatment and management of animals with immune-mediated diseases.

Dosage and Administration: SR-Rapamycin is a drug formulation to be administered via injection, such as a subcutaneous (SQ) injection. Subcutaneous injections in humans are typically given in areas with fatty tissues like the abdomen (away from the naval), outer thighs, back of the upper arms, and upper buttocks. Subcutaneous injections in animals other than humans are typically given in areas with loose skin, such as the neck, shoulders, or flank. Sites for subcutaneous injections include the outer lateral aspect of the upper arm, the abdomen (from below the costal margin to the iliac crest and more than two inches from the umbilicus), the anterior upper thighs, the upper back, and the upper ventral gluteal area. The most usual site for injection is over the shoulders, into the loose skin over the neck, but other sites with loose skin can also be used, for example the flank. When repeated doses of material are needed, varying the site of injection can help reduce the likelihood of local skin reactions.

Dogs: In the back of the neck or “scruff” region (between the shoulder blades). Pinch some loose skin between one's thumb and forefinger.

Cats: The most common injection sites include: abdomen—at or under the level of the bellybutton, about 2 inches away from the navel; Arm—back or side of the upper arm; and Thigh—front of the thigh.

Administration: SR-Rapamycin may be a liquid, suspension, semi-solid gel, or solid implant that releases rapamycin over time. In the microsphere embodiments, the microspheres will be in a suspension.

Duration of dosing: The sustained release drug product may release rapamycin for days (1, 2, 3, 4, 5, 6, 7, etc.), weeks (1, 2, 3, 4, etc.), months (1, 2, 3, 4, 5, 6, etc.), or as much as a year.

Dosage: The therapeutic amount of drug released from the sustained release formulation may be based on the animal's body weight in kilograms (kg) or pounds (lb). The preferred amount of drug released into the animal may be based on, or stated as, milligrams per kg (mg of the drug/kg body weight) or micrograms per kg (ug/kg).

In some embodiments, the range of daily drug product (rapamycin and/or rapamycin analogs) released from the sustained release formulation may range from 0.001 μg/kg/day to 5000 ug/kg/day (5 mg of drug per kg of the subject's (animal) body weight per day). A desired dose range for humans or animals with chronic, residual disease may be between 0.1 to 500 μg/kg/day. The range of doses to be administered in one subcutaneous injection to cats or dogs to deliver sustained therapeutic levels of rapamycin over time may range from 0.01 to 60 mg/kg of the subject's body weight. A therapeutically effective blood level of the drug may range from 1 ng/ml (nanogram per milliliter) blood to 50 ng/mL blood.

The dose of the drug that is administered to an animal may vary according to the volume of the formulation that is injected into the subject. For example, a 1 to 6 mL syringe may be used to administer the drug. Without limitation, a dosage of drug administration may vary from 0.1 mL to 6 mL depending on the subject's body weight. Doses larger than 2 mL may be divided into equal fractions and administered as 2 immediate sequential doses.

Upon initial injection, the drug product may release the drug in an initial burst, followed by a leveling off of drug levels in the blood, then followed by a tapering of drug levels over time.

Drug Holidays: Associated with rapamycin's potent immunosuppressive therapeutic activities, some animals may need a drug holiday from SR-Rapamycin therapy. Over treatment with rapamycin may be associated with suppression of cellular growth and multiplication that could result in a lowering of the immune system that could result in cellular anemias (low blood cell counts), or clinical pneumonia. Therefore, during the course of treatment of a chronic, immune-mediated disease, breaks in drug administration may be recommended or needed. For example, if the sustained-release drug product lasts 1 or 2 weeks, a drug holiday of 3 to 5 days may be needed in between consecutive administrations of the drug. If the sustained-release drug product lasts one month, a drug holiday of 1-2 weeks may be needed in between doses. If the drug product lasts six months or one year, a drug holiday of one month may be needed.

Injections: Subcutaneous injections may be directed into the fat tissue just below the skin. For humans, the needle required is small and short, typically ½ to ⅝ of an inch with a gauge of 25 to 30. However, a 20-gauge needle may be used for cats and small dogs, while an 18-gauge needle may be used for larger pets. For cats, a 20-gauge or 22-gauge needle is most commonly used. Without limitation, a large dog may need a 22-gauge 1 inch needle while a small cat may need a 25-gauge, ⅝ inch needle.

### Animal Target Product Profile.

TABLE 4
Summary of One Targeted Pharmaceutical Product and Parameters
for Administering the Pharmaceutical Product to a Cat or Dog
Animal target product profile.

| Active Pharmaceutical Ingredient | | Rapamycin/Sirolimus (MWT: 914 g/mole) | |
|---|---|---|---|
| Target Indications | | Veterinary Immunology - Antibiotic Refractory | |
| | | Gingivitis, IBD, Atopic Dermatitis | |
| Dosage Form | | Sustained-release injectable | |
| Total dose (cat) - given once for 2 weeks. | | 36 mg (range: 24-72 mg) active ingredient | |
| The dose may be repeated every 2 to | | | |
| 4 weeks as needed to induce remission. | | | |
| Daily Dose | | ~2.5 mg per day | |
| In Vitro Release Profile | | Sustained release for two weeks with zero order release | |
| | | kinetics. A burst with a steady decline is not desired. A | |
| | | continuous/even delivery over a 2 week period is | |
| | | desired. Some tailing of the release is expected. | |
| Route of Administration | | Subcutaneous injection | |
| Injection Frequency | | Once every two to four weeks, with a possible break for | |
| | | 7 to 14 days between doses as needed for safety to | |
| | | avoid inducing toxic immunosuppression. | |
| PK blood levels desired | | 24-hr (trough) Range: 5-15 ng Rapamycin/mL has | |
| | | been the historical oral goal. Safety pK studies are to | |
| | | be performed to determine a maximum tolerable dose | |
| | | (MTD), then will back off to a lower dose. | |
| Safety dose to study with diseased subjects | | Dose that does not induce cytopenia | |
| Injection volume | Cats: 1-2 mL | | Dogs: possibly 1-6 mL which will |
| | | | be weight dependent |
| Syringe size | 25 g syringe | Range: 22-25 g | Cat or Dog |
| Rapamycin content | 20% wt | | |
| Burst release | Some allowed ~5% | A small loading | |
| | | dose is ok. | |

### Immune-Mediated, Inflammatory and Autoimmune Diseases

Some embodiments provide a method of treating one or more immune-mediated, inflammatory and autoimmune diseases of the animal, which specifically includes veterinary animals and/or humans. The one or more immune-mediated, inflammatory disease may be selected from rheumatoid arthritis, polymyalgia rheumatica, psoriatic arthritis, immune-mediated polyarthritis rheumatism unspecified, ankylosing spondylitis, spondylarthrites, rheumatic fever or rheumatic heart diseases, arthritis associated to inflammatory bowel disease, inflammatory bowel disease, ulcerative colitis, celiac disease, Crohn's disease, autoimmune gastritis, primary biliary cholangitis, irritable bowel syndrome (IBD), feline idiopathic interstitial cystitis (FIC), cysts, adenomas, polyps, lymphoplasmacytic gastritis, autoimmune hepatitis, primary biliary cirrhosis, primary biliary cholangitis, primary sclerosing cholangitis, acute hemorrhagic diarrhea syndrome, immune-mediated glomerulonephritis, chronic or cyclic vomiting syndrome (CVS), Kawasaki disease, diabetes (type 1), hyperthyroidism, autoimmune hypothyroidism, lymphocytic thyroiditis, Graves' disease or autoimmune thyroiditis uveitis, uveodermatologic syndrome, Guillain-Barre syndrome, myasthenia gravis, Addison's disease, atopic dermatitis, eczema, psoriasis, skin psoriasis, erythema nodosum, erythema multiforme, epidermolysis bullosa acquisita, autoimmune subepidermal blistering syndrome, systemic lupus erythematosus, discoid lupus erythematosus, cutaneous vasculitis, mucocutaneous lupus erythematosus, vesicular cutaneous lupus erythematosus, sterile nodular dermatitis and panniculitis, symmetric lupoid onychitis, juvenile cellulitis, uveodermatological syndrome, toxic epidermal necrolysis, Stevens-Johnson syndrome, Sweet's-like syndrome, Wells-like syndrome, bullous disorders, bullous pemphigoid, pemphigus foliaceus, pemphigus erythematosus, pemphigus vulgaris, pemphigus vegetans, pemphigus complex, paraneoplastic pemphigus, autoimmune diseases of the pineal gland, autoimmune and immune-mediated cellulitis, juvenile cellulitis, Hidradenitis, Lichen planus, laminitis, stomatitis, gingivitis, gingivostomatitis, feline chronic gingivostomatitis (FCGS), periodontal disease, periodontitis, pulpitis, feline odontoclastic resorption lesions (FORL), asthma, allergic rhinitis, chronic rhinitis, feline lymphoplasmacytic rhinitis (FLPCR), rhinotracheitis, sinusitis, chronic idiopathic rhinosinusitis, nasal arteritis, nasal vestibulitis, autoimmune pulmonary alveolar proteinosis, chronic bronchitis, Sicca syndrome, Sjogren's syndrome, pulmonary hypertension, interstitial lung disease (IDL), mixed connective tissue disease of the lung, sarcoidosis, neurosarcoidosis myocarditis, rheumatic heart disease, hypertrophic cardiomyopathy, enlarged heart, cardiomegaly, autoimmune-related liver sarcoidosis, pericarditis, endocarditis, coronary artery disease, Berger's disease-IgA nephropathy, membranous nephropathy, renal fibrosis, renal scarring, lupus nephritis, anti-neutrophil cytoplasmic associated vasculitis (AAV), Goodpasture's disease/anti-glomerular basement disease, glomerulonephritis, multiple sclerosis, myositis, polymyositis, masticatory muscle myositis, dermatomyositis, scleroderma, neuromyelitis optica spectrum disorders, autoimmune encephalitis, transverse myelitis, optic neuritis, acute disseminated encephalomyelitis, granulomatous meningoencephalomyelitis (GME), meningoencephalomyelitis of unknown origin (MUO), eosinophilic meningoencephalomyelitis, steroid-responsive meningitis-arteritis (SRMA), necrotizing encephalitia (NE), autoimmune related epilepsy, central nervous system vasculitis, myelin oligodendrocyte glycoprotein antibody disease (MOG), para neoplastic disorders of the central nervous system, chronic meningitis, Evans syndrome, immune-mediated neutropenia, immune-mediated thrombocytopenia, antiphospholipid syndrome, autoimmune myelofibrosis, acquired aplastic anemia, paroxysmal nocturnal hemoglobinuria, hypoplastic myelodysplastic syndromes, large granular lymphocyte leukemia, immune-mediated hemolytic anemia (IMHA), immune-mediated vasculitis, somatic or enteropathic arthropathies, necrotizing vasculopathies, idiopathic thrombocytopenia purpura, feline immunodeficiency virus (FIV), feline leukemia virus (FeLV), feline foamy virus (FFV), cytomegalovirus, herpesvirus, and calicivirus.

Diseases Associated with Accumulated Debris, Fibrosis or Antigenic Particles.

Some embodiments provide a method of treating one or more diseases associated with a buildup of cellular debris, fibrosis, or antigen load of a subject (animal) in need of a therapeutic aid. Rapamycin's ability to stimulate autophagy—the cell's internal recycling and cleanup system—may have therapeutic benefits in a range of human or veterinary diseases affecting animals, specifically including veterinary animals and/or humans, where damaged proteins, dysfunctional organelles, accumulation of fibrotic, antigenic materials, or chronic inflammation play a central role.

In immune-mediated diseases rapamycin, via autophagy, may help in the removal of sequestered antigenic particles such as intact or segments of viral or bacterial invaders. Rapamycin may also be beneficial by helping to close open barriers in tissues such as skin, the largest immune organ, that can occur in atopic dermatitis, thus not allowing more invasive antigens to penetrate into skin or other organs and therefore decreasing the antigenic load.

In neurodegenerative conditions such as cognitive dysfunction syndrome in older animals, such as humans and/or veterinary species such as dogs and cats, enhanced autophagy may help clear misfolded proteins and support healthier neuronal function. Similarly, in cardiomyopathies and age-related heart disease, rapamycin may antagonize overgrowth and enlargement of cardiac cells, and via autophagy may improve cellular energy balance and remove damaged mitochondria, potentially supporting better cardiac resilience. Rapamycin's autophagy-boosting effects may also be of benefit in treating chronic kidney disease, where clearing cellular debris and fibrotic materials may slow progression of tubular damage. In metabolic disorders, including obesity-related inflammation or insulin-resistant states, autophagy helps restore healthier cellular signaling pathways. Even certain cancers may be influenced, as autophagy can either suppress early tumor development by removing damaged components or modulate how cancer cells respond to stress. The unifying theme is that rapamycin's activation of autophagy may help cells maintain stability and function in diseases driven by accumulated damage or chronic stress.

### Role of Rapamycin in Autophagy

Rapamycin induces autophagy by inhibiting a central cellular growth regulator called mTORC1 (mechanistic target of rapamycin complex 1). Under normal conditions, mTORC1 suppresses autophagy by blocking the activation of proteins required to initiate autophagosome formation. When rapamycin binds to the protein FKBP12, the resulting complex directly inhibits mTORC1, lifting this suppression. As mTORC1 activity falls, upstream autophagy initiating kinases such as ULK1 become activated, allowing the cell to begin assembling the autophagy machinery. This shift promotes the formation of autophagosomes, enhances lysosomal biogenesis, and ultimately increases the degradation and recycling of cellular components. In essence, rapamycin pushes the cell into a resource conserving, self-cleaning mode by turning down growth signals and turning up catabolic pathways.

Many animal diseases involve the buildup of excessive cellular debris, such as damaged proteins, dysfunctional organelles, or waste material that cells fail to clear efficiently, and this accumulation often contributes to chronic inflammation or tissue degeneration. Rapamycin provides a dose-dependent response that allows for controlled autophagy induction. Furthermore, the effect of rapamycin on autophagy levels is reversible when rapamycin is removed. Accordingly, rapamycin is a powerful tool for studying autophagy and a promising therapeutic agent for diseases involving defective cellular quality control.

Rapamycin's interaction with mTORC2 is more nuanced than its well-known inhibition of mTORC1. In most cell types, rapamycin does not acutely inhibit mTORC2. Rather, rapamycin interferes with mTORC2 more gradually. Rapamycin binds FKBP12 and, over prolonged exposure, this complex prevents the proper assembly of new mTORC2 complexes. As a result, mTORC2 activity declines over time, reducing phosphorylation of key downstream targets such as AKT at Ser473. Because mTORC2 normally supports cell survival, cytoskeletal organization, and metabolic regulation, its inhibition can shift cells toward reduced growth signaling and altered stress responses. This longer-term suppression of mTORC2 is thought to contribute to some of rapamycin's broader physiological effects, including potential impacts on insulin sensitivity and metabolic pathways.

mTORC2's involvement in rapamycin's long-term side effects comes from the fact that this complex helps regulate how cells handle metabolism, insulin signaling, and survival pathways. Rapamycin does not strongly inhibit mTORC2 at first, but with chronic exposure it can prevent new mTORC2 complexes from forming. As mTORC2 activity gradually declines, cells lose some of their ability to activate AKT at Ser473, which is a modification important for maintaining insulin responsiveness and balanced glucose metabolism. This shift is thought to underlie rapamycin's association with insulin resistance or glucose intolerance in some long-term treatment settings. Because mTORC2 also supports cytoskeletal organization and stress-response pathways, its suppression may contribute to broader metabolic or tissue-specific effects seen with extended therapy. In short, while rapamycin's benefits are tied to mTORC1 inhibition, some of its long-term side effects likely stem from the slower, secondary disruption of mTORC2-dependent signaling.

Treating animals with an injectable form of rapamycin can offer several practical and biological advantages compared with giving the drug as a tablet. In many species, oral rapamycin has variable absorption, meaning the amount that actually reaches the bloodstream can fluctuate depending on the animal's gut physiology, diet, and/or stress level. An injectable formulation bypasses the digestive system entirely, providing more predictable and consistent drug exposure. This can be especially valuable in research or veterinary settings where stable dosing is important. Injections also avoid issues with poor palatability, where some animals may resist or refuse tablets, making long-term oral treatment unreliable. Additionally, injectable delivery can reduce the influence of first-pass metabolism in the liver, potentially improving bioavailability and allowing lower or less frequent dosing. Altogether, these factors can make injectable rapamycin a more controlled and dependable way to achieve therapeutic levels in animals.

Rapamycin's ability to stimulate autophagy (the cell's internal recycling and cleanup system) may provide a benefit in treating a range of canine and feline diseases where damaged proteins, dysfunctional organelles, or chronic inflammation play a central role. For example, in neurodegenerative conditions such as cognitive dysfunction syndrome in older dogs and cats, enhanced autophagy may help clear misfolded proteins and support healthier neuronal function. Similarly, in cardiomyopathies and age-related heart disease, autophagy can improve cellular energy balance and remove damaged mitochondria, potentially supporting better cardiac resilience. Rapamycin's autophagy-boosting effects may also provide benefit in chronic kidney disease, where clearing cellular debris may slow progression of tubular damage. In diseases involving invasive bacteria, viruses, or environmental antigens the activated autophagy process may serve to remove bacterial, viral or other antigenic particles located inside cells. In metabolic disorders, including obesity-related inflammation or insulin-resistant states, autophagy may help restore healthier cellular signaling pathways. Some liver diseases, including hepatic lipidosis in cats or chronic hepatitis in dogs, feature excessive intracellular fat or protein aggregates that overwhelm normal cellular cleanup pathways. Even certain cancers may be influenced, as autophagy can either suppress early tumor development by removing damaged components or modulate how cancer cells respond to stress. However, rapamycin's activation of autophagy may help cells maintain stability and function in diseases driven by accumulated damage or chronic stress.

The terminology used herein is for the purpose of describing particular embodiments only and is not intended to limit the scope of the claims. As used herein, the singular forms “a”, “an” and “the” are intended to include the plural forms as well, unless the context clearly indicates otherwise. It will be further understood that the terms “comprises”, “comprising”, “includes” and/or “including” when used in this specification, specify the presence of stated features, integers, steps, operations, elements, components and/or groups, but do not preclude the presence or addition of one or more other features, integers, steps, operations, elements, components, and/or groups thereof. The terms “preferably,” “preferred,” “prefer,” “optionally,” “may,” and similar terms are used to indicate that an item, condition or step being referred to is an optional (not required) feature of the embodiment.

The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

When reference is made to the term “each,” it is not meant to mean “each and every, without exception.” For example, if reference is made to microsphere formulation comprising polymer microspheres, and “each polymer microsphere” is said to have a particular active pharmaceutical ingredient (API) content, if there are 10 polymer microspheres, and two or more of the polymer microspheres have the particular API content, then that subset of two or more polymer microspheres is intended to meet the limitation.

The term “about” in conjunction with a number is simply shorthand and is intended to include ±10% of the number. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

The corresponding structures, materials, acts, and equivalents of all means or steps plus function elements in the claims below are intended to include any structure, material, or act for performing the function in combination with other claimed elements as specifically claimed. Embodiments have been presented for purposes of illustration and description, but it is not intended to be exhaustive or limited to the embodiments in the form disclosed. Many modifications and variations will be apparent to those of ordinary skill in the art after reading this disclosure. The disclosed embodiments were chosen and described as non-limiting examples to enable others of ordinary skill in the art to understand these embodiments and other embodiments involving modifications suited to a particular implementation.

What is claimed is:

1. A microsphere formulation, comprising:
polymer microspheres, each polymer microsphere comprising:
an active pharmaceutical ingredient consisting of rapamycin, a rapamycin analog, a pharmaceutically acceptable salt of rapamycin, and/or a pharmaceutically acceptable salt of the rapamycin analog; and
a biodegradable polymer, wherein each polymer microsphere comprises a drug load of the active pharmaceutical ingredient that is between 20 and 40 percent by weight of the polymer microsphere, and wherein the polymer microspheres have a median particle size of about 15 micrometers to about 30 micrometers.

2. The microsphere formulation of claim 1, wherein the biodegradable polymer comprises a poly(lactide-co-glycolide) (PLGA) polymer.

3. The microsphere formulation of claim 2, wherein the PLGA polymer has a lactide:glycolide ratio of about 50:50.

4. The microsphere formulation of claim 3, wherein the biodegradable polymer is acid-terminated.

5. The microsphere formulation of claim 3, wherein the biodegradable polymer has an inherent viscosity between about 0.1 and about 0.3 deciliters per gram.

6. The microsphere formulation of claim 3, wherein the biodegradable polymer has an inherent viscosity of about 0.12 deciliters per gram.

7. The microsphere formulation of claim 1, wherein the polymer microspheres have a median particle size of about 30 micrometers.

8. The microsphere formulation of claim 1, wherein the drug load of the active pharmaceutical ingredient is about 25 percent by weight of the polymer microspheres.

9. The microsphere formulation of claim 1, characterized by a sustained release of the active pharmaceutical ingredient over a period ranging from about 14 days to about 30 days.

10. The microsphere formulation of claim 1, characterized by a sustained release of the active pharmaceutical ingredient over a period ranging from about 14 days to about 21 days.

11. The microsphere formulation of claim 1, wherein the polymer microspheres have been irradiated.

12. The microsphere formulation of claim 1, wherein the active pharmaceutical ingredient consists essentially of rapamycin.

13. The microsphere formulation of claim 12, wherein the active pharmaceutical ingredient contains less than one percent seco-rapamycin.

14. The microsphere formulation of claim 1, characterized in that more than 75% of the active pharmaceutical ingredient is released over a period of about 21 days following injection into a subject and less than 20% of the active pharmaceutical ingredient is released within about 24 hours following injection into the subject.

15. A pharmaceutical composition comprising the microsphere formulation of claim 1 and a diluent.

16. A therapeutic regimen for treating an immune-mediated disease in a subject using the microsphere formulation of claim 1, comprising:
administering an injection of a pharmaceutical composition comprising the microsphere formulation and a diluent, wherein the pharmaceutical composition provides a therapeutically effective blood concentration of the active pharmaceutical ingredient greater than 5 nanograms per milliliter of blood of the subject over a period of at least about 14 days.

17. The therapeutic regimen of claim 16, wherein the pharmaceutical composition is administered to the subject with a dosing schedule of about every 14 to 30 days.

18. The therapeutic regimen of claim 17, wherein the dosing schedule is about every 14 to 21 days.

19. The therapeutic regimen of claim 17, further comprising:
implementing a drug-holiday period between doses.

20. A kit, comprising:
a first container including the microsphere formulation of claim 1; and
a second container including a diluent to be added to the microsphere formulation to form a pharmaceutical composition for administering to a subject via injection.
